(19) United States
(12) Patent Application Publication
(10) Pub. No.: US 2023/0398529 A1
(43) Pub. Date: Dec. 14, 2023

(54) ASYMMETRIC TRANSFER HYDROGENATION OF 2-ARYL SUBSTITUTED BICYCLIC PYRIDINE KETONES IN PRESENCE OF A CHIRAL RUTHENIUM CATALYST

(71) Applicant: Bayer Aktiengesellschaft, Leverkusen (DE)

(72) Inventors: Christoph SCHOTES, Duesseldorf (DE); Sébastien GOUDEDRANCE, Sierentz (FR); Felix SPINDLER, Starrkirch-Wil (CH); Beat FUCHS, Kriens (CH)

(21) Appl. No.: 18/248,535

(22) PCT Filed: Oct. 11, 2021
(86) PCT No.: PCT/EP2021/078025

§ 371 Date: Apr. 11, 2023

(30) Foreign Application Priority Data
Oct. 13, 2020 (EP) 20201628.3

(51) Int. Cl. B01J 31/22; B01J 31/18; C07D 221/04

(52) U.S. Cl. CPC B01J 31/2295; B01J 31/2217; B01J 31/182; C07D 221/04; B01J 2531/821; B01J 2531/004; B01J 2231/643

(57) ABSTRACT

The invention relates to a process for preparing optically active 2-aryl substituted 6,7-dihydro-5H-cyclopenta[b]pyridin-7-ols comprising asymmetric transfer hydrogenation of the corresponding ketones in presence of a ruthenium catalyst comprising a chiral diamine or amino alcohol ligand.

The invention relates to a process for preparing optically active 2-aryl substituted 6,7-dihydro-5H-cyclopenta[b]pyridin-7-ols comprising asymmetric transfer hydrogenation of the corresponding ketones in presence of a ruthenium catalyst comprising a chiral diamine or amino alcohol ligand.

It is known from WO 2019/185541 that chiral aryl-substituted bicyclic pyridine-phosphinites are excellent (P,N)-ligands for the iridium-catalyzed enantioselective hydrogenation of 4-substituted N-acetyl-dihydroquinolines. By means of these ligands, the resulting 4-substituted N-acetyl-tetrahydroquinolines can be obtained in high yields and excellent enantioselectivity (up to 98% ee). Subsequent rearrangement gives access to the corresponding 4-aminoindane derivatives (EP 0 654 464), which are important intermediates for the preparation of various N-indanyl heteroaryl carboxamides having fungicidal activity (EP 0 654 464, WO 2011/162397, WO 2012/084812, WO 2015/197530).

Chiral aryl-substituted bicyclic pyridine-phosphinites can be prepared via resolution of racemic aryl-substituted 6,7-dihydro-5H-cyclopenta[b]pyridin-7-ols using chiral HPLC and conversion to the corresponding phosphinites by deprotonation and subsequent treatment with di(cyclo)alkyl chlorophosphane (S. Kaiser et al., Angew. Chem. Int. Ed. 2006, 45, 5194-5197). A kinetic resolution of the racemic alcohols with lipase or by copper-catalyzed benzoylation is also known (D. H. Woodmansee et al., Chem. Sci. 2010, 1, 72-78; C. Mazet et al., Org. Lett. 2006, 8, 1879-1882). However, a general disadvantage of racemic resolution methods is that the desired and undesired enantiomers are always obtained in equal amounts which requires additional steps to convert the undesired enantiomer to the desired enantiomer, e.g. by repeated oxidation and racemic resolution sequences.

In principle, methods for asymmetric reduction of bicyclic pyridine ketones are also already available. An asymmetric transfer hydrogenation of bicyclic pyridine ketones in presence of a chiral iron catalyst is known from A. Naik et al., Chem. Commun. 2010, 46, 4475-4477. However, it was found that a substituent in 2-position is detrimental to the enantioselectivity, and accordingly 2-aryl substituted bicyclic pyridine alcohols could only be obtained with moderate enantioselectivities (52-72% ee) Similarly, a catalytic enantioselective reduction of 2-phenyl-6,7-dihydro-5H-quinolin-8-one with (S)-Me-CBS-borane was reported to afford the corresponding ketone with an enantiomeric excess of 72% only (Tetrahedron: Asymmetry, 2009, 20, 1425-1432).

In the light of the prior art described above, it is an object of the present invention to provide a process for preparing optically active 2-aryl substituted 6,7-dihydro-5H-cyclopenta[b]pyridin-7-ol derivatives which process has advantages over the processes of the prior art. In particular, the process should allow the desired enantiomer to be prepared in high yield and high enantiomeric purity.

The object described above was achieved by a process for preparing a compound of the formula (Ia) or (Ib),

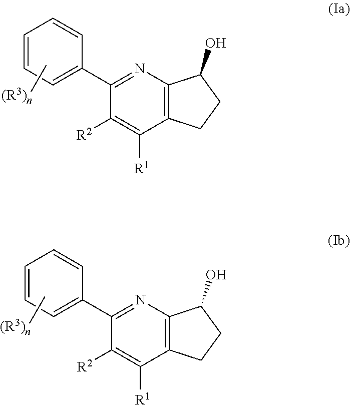

where
R¹ and R² are independently from one another selected from the group consisting of hydrogen and C₁-C₄-alkyl,
each R³, if present, is independently selected from C₁-C₄-alkyl, and
n is 0, 1, 2 or 3,
comprising asymmetric transfer hydrogenation of a ketone of the formula (II)

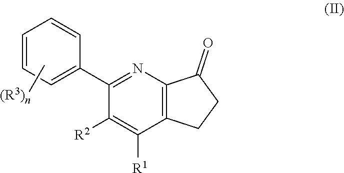

in which the substituents R¹, R², R³ and the integer n are as defined for the compound of the formula (Ia) or (Ib),
in presence of a chiral ruthenium catalyst and a polar solvent, wherein the ruthenium catalyst comprises a chiral amino alcohol ligand or a chiral diamine ligand.

It has been found, surprisingly, that optically active 2-aryl-substituted 6,7-dihydro-5H-cyclopenta[b]pyridin-7-ol derivatives (of the formulae Ia and Ib) can be prepared in high yields and excellent enantioselectivity by asymmetric transfer hydrogenation of the corresponding 2-aryl substituted bicyclic pyridine ketones (of the formula II) in presence of a chiral ruthenium catalyst comprising a chiral amino alcohol or diamine as ligand.

### Definitions

In the definitions of the symbols given in the formulae above and below, collective terms were used, which are generally representative of the following substituents:

The term “halogen” as used herein refers to fluorine, chlorine, bromine or iodine atom.

The term “C₁-C₄-alkyl” as used herein refers to a saturated, branched or straight hydrocarbon chain having 1, 2, 3 or 4 carbon atoms. Examples of C₁-C₄-alkyl include methyl, ethyl, propyl (n-propyl), 1-methylethyl (iso-propyl), butyl (n-butyl), 1-methylpropyl (sec-butyl), 2-methylpropyl (iso-butyl) and 1,1-dimethylethyl (tert-butyl).

The term “C₂-C₆-alkyl” as used herein refers to a saturated, branched or straight hydrocarbon chain having 2, 3, 4, 5 or 6 carbon atoms. Examples of C₂-C₆-alkyl include but are not limited to ethyl, propyl (n-propyl), 1-methylethyl (iso-propyl), butyl (n-butyl), 1-methylpropyl (sec-butyl), 2-methylpropyl (iso-butyl), 1,1-dimethylethyl (tert-butyl), pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The terms “phenyl-(CH₂)₃—”, “phenyl-(CH₂)₄—” and “phenyl-(CH₂)₂—O—CH₂—” as used herein refer to a phenyl group, which is unsubstituted or substituted as defined herein and which is attached to the parent moiety via a —(CH₂)₃—, —(CH₂)₄— or —(CH₂)₂—O—CH₂— linker.

As used herein, when a group is said to be “substituted”, the group may be substituted with one or more substituents where the substituents may be identical or different. The expression “one or more substituents” refers to a number of substituents that ranges from one to the maximum number of substituents possible based on the number of available bonding sites, provided that the conditions of stability and chemical feasibility are met.

The term “enantioselective” as used herein means that one of the two possible enantiomers of the hydrogenation product, namely the enantiomer of the formula (Ia) (or (Ia′)) or the enantiomer of the formula (Ib) (or (Ib′)), is preferably formed. The “enantiomeric excess” or “ee” indicates the degree of enantioselectivity:

$$\% ee = \frac{major enantiomer ( mol ) - minor enentiomer ( mol )}{major enantiomer ( mol ) + minor enentiomer ( mol )} \times 100 \%$$

The major enantiomer can be controlled by the selection of the chiral ligand, for example by selecting the chiral ligand of the formula (IIIa) or the opposite enantiomer (the ligand of the formula (IIIb)), or by selecting the chiral ligand of the formula (IVa) or the opposite enantiomer (the ligand of the formula (IVb)).

The process according to the invention is used for preparing the compound of the formula (Ia) or (Ib), preferably (Ia), using a compound of the formula (II) as starting material.

Preferred are compounds of the formulae (Ia), (Ib) and (II), in which R² is H.

More preferred compounds of the formula (Ia), (Ib) and (II) are compounds of formulae (Ia′), (Ib′) and (II′)

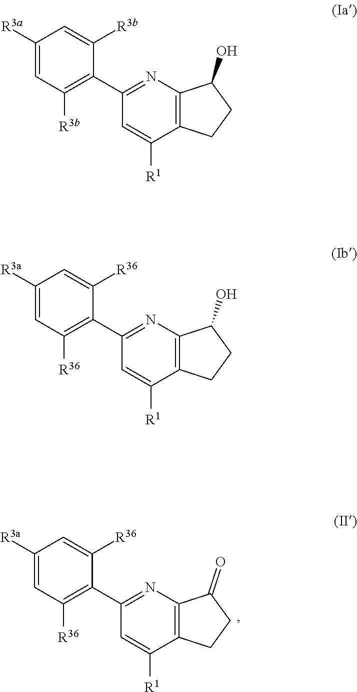

wherein R¹, R^{3a} and R^{3b} are independently of one another selected from C₁-C₄-alkyl.

Even more preferred are compounds of the formulae (Ia), (Ib) and (II) and compounds of the formulae (Ia′), (Ib′) and (II′), in which R¹ is methyl.

Particularly preferred are compounds of the formulae (Ia′), (Ib′) and (II′), in which
R¹ is methyl,
R^{3a} is methyl, and
R^{3b} is ethyl.

The process according to the invention comprises asymmetric transfer hydrogenation of the compound of the formula (II), preferably (II′). The substituents R¹, R² and R³ and the integer n in the compound of the formula (II) are each as defined for the compounds of the formulae (Ia) and (Ib). Accordingly, the substituents R¹, R^{3a} and R^{3b} in the compound of the formula (II′) are each as defined for the compounds of the formulae (Ia′) and (Ib′).

The asymmetric transfer hydrogenation of the compound of the formula (II) is conducted in the presence of a chiral ruthenium catalyst comprising a chiral amino alcohol ligand or a chiral diamine ligand.

Preferably, the chiral ruthenium catalyst comprises a chiral amino alcohol or diamine ligand of the formula (IIIa) or (IIIb) or a chiral amino alcohol ligand of the formula (IVa) or (IVb)

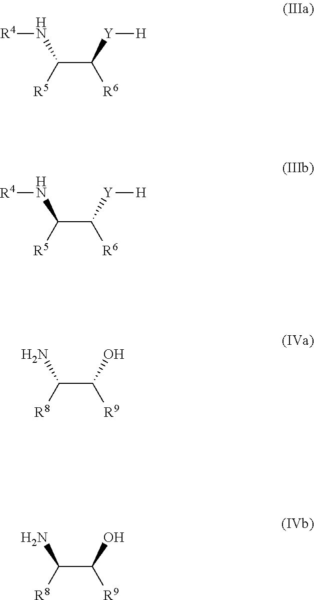

where
Y is NR⁷ or O,
R⁴ is phenylsulphonyl, wherein the phenyl is unsubstituted or substituted with one or more substituents independently selected from C₁-C₄-alkyl and halogen, or
R⁴ is 2-pyrrolidinylcarbonyl or 2-piperidinylcarbonyl, preferably (2S)-2-pyrrolidinylcarbonyl,
R⁵ and R⁶ together form a —(CH₂)₃— or —(CH₂)₄— group, or
R⁵ and R⁶ are independently of one another selected from phenyl, which is unsubstituted or substituted with one or more substituents independently selected from C₁-C₄-alkyl,
R⁷ is hydrogen, phenyl-(CH₂)₃—, phenyl-(CH₂)₄—, benzyloxymethyl, benzyloxyethyl or phenyl-(CH₂)₂—O—CH₂—, wherein the phenyl and benzyl groups are optionally substituted with one or more substituents independently selected from C₁-C₄-alkyl,
R⁸ is C₂-C₆-alkyl and R⁹ is hydrogen, or
R⁸ and R⁹ are independently of one another selected from phenyl, which is unsubstituted or substituted with one or more substituents independently selected from C₁-C₄-alkyl, or
R⁸ and R⁹ form together a group of the formula

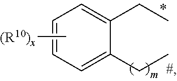

where the bond marked with “*” is connected to the carbon bearing the hydroxyl group and the bond marked with “#” is connected to the carbon bearing the amino group, and wherein
m is 0 or 1,
x is 0, 1 or 2, and
each R¹⁰, if present, is independently selected from C₁-C₄-alkyl.

Depending on whether compound (Ia) or (Ib) is the desired product, a ligand of the formula (IIIa) or (IVb) or a ligand of the formula (IIIb) or (IVa) is suitable for use in the process according to the invention. Usually, if a compound of the formula (Ia) is the desired product, a ligand of formula (IIIa) or (IVb), preferably (IIIa), is suitable for use in the process according to the invention, whereas if a compound of the formula (Ib) is the desired product, a ligand of formula (IIIb) or (IVa), preferably (IIIb), is suitable for use in the process according to the invention.

More preferably, the chiral ruthenium catalyst comprises a chiral ligand of the formula (IIIa), (IIIb), (IVa) or (IVb), where
Y is NR⁷ or O,
R⁴ is phenylsulphonyl, wherein the phenyl is unsubstituted or substituted with one or more substituents independently selected from C₁-C₄-alkyl and halogen, or
R⁴ is 2-pyrrolidinylcarbonyl, preferably (2S)-2-pyrrolidinylcarbonyl,
R⁵ and R⁶ together form a —(CH₂)₄— group, or
R⁵ and R⁶ are unsubstituted phenyl,
R⁷ is hydrogen, phenyl-(CH₂)₃—, phenyl-(CH₂)₄—, benzyloxymethyl, benzyloxyethyl or phenyl-(CH₂)₂—O—CH₂— group, wherein the phenyl and benzyl groups are optionally substituted with one or more substituents independently selected from C₁-C₄-alkyl,
R⁸ and R⁹ are unsubstituted phenyl, or
R⁸ and R⁹ form together a group of the formula

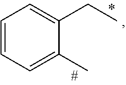

wherein the bond marked with “*” is connected to the carbon bearing the hydroxyl group and the bond marked with “#” is connected to the carbon bearing the amino group.

Most preferred are chiral ligands of the formula (IIIa) or (IIIb), where
Y is NR⁷ or O,
R⁴ is phenylsulphonyl, wherein the phenyl is unsubstituted or substituted with one or more substituents independently selected from C₁-C₄-alkyl and halogen, or
R⁴ is 2-pyrrolidinylcarbonyl, preferably (2S)-2-pyrrolidinylcarbonyl,
R⁵ and R⁶ together form a —(CH₂)₄— group, or
R⁵ and R⁶ are unsubstituted phenyl,
R⁷ is hydrogen, phenyl-(CH₂)₃—, phenyl-(CH₂)₄—, benzyloxymethyl, benzyloxyethyl or phenyl-(CH₂)₂—O—CH₂— group, wherein the phenyl and benzyl groups are optionally substituted with one or more substituents independently selected from C₁-C₄-alkyl.

Particularly preferred are chiral diamine ligands of the formula (IIIa-1) or (IIIb-1)

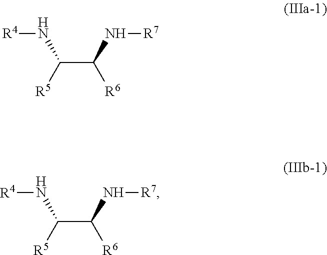

in which
R⁴ is phenylsulphonyl, wherein the phenyl is unsubstituted or substituted with one or more substituents independently selected from C₁-C₄-alkyl and fluorine,
R⁵ and R⁶ together form a —(CH₂)₄— group, or
R⁵ and R⁶ are unsubstituted phenyl, and
R⁷ is hydrogen, phenyl-(CH₂)₃—, phenyl-(CH₂)₄—, benzyloxymethyl, benzyloxyethyl or phenyl-(CH₂)₂—O—CH₂— group, wherein the phenyl and benzyl groups are optionally substituted with one or more substituents independently selected from C₁-C₄-alkyl,
preferably R⁷ is hydrogen, phenyl-(CH₂)₃— or benzyloxyethyl, wherein the phenyl and benzyl groups are optionally substituted with one or more substituents independently selected from C₁-C₄-alkyl, preferably methyl;
and chiral amino alcohol ligands of the formula (IIIa-2) or (IIIb-2), where

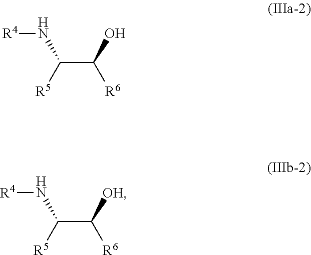

R⁴ is 2-pyrrolidinylcarbonyl, preferably (2S)-2-pyrrolidinylcarbonyl, and
R⁵ and R⁶ together form a —(CH₂)₄— group, or
R⁵ and R⁶ are unsubstituted phenyl,
preferably R⁵ and R⁶ are unsubstituted phenyl.

Ligands of the formulae (IIIa), (IIIb), (IVa) and (IVb) are commercially available or may be prepared by methods known in the art (e.g. R. Hodgkinson et al., Organometallics, 2014, 33, 5517-5524; V. Parekh et al., Catal. Sci. & Technol., 2012, 2, 406-414).

Preferably, the chiral ruthenium catalyst has the general formula (Va), (Vb), (VIa) or (VIb):

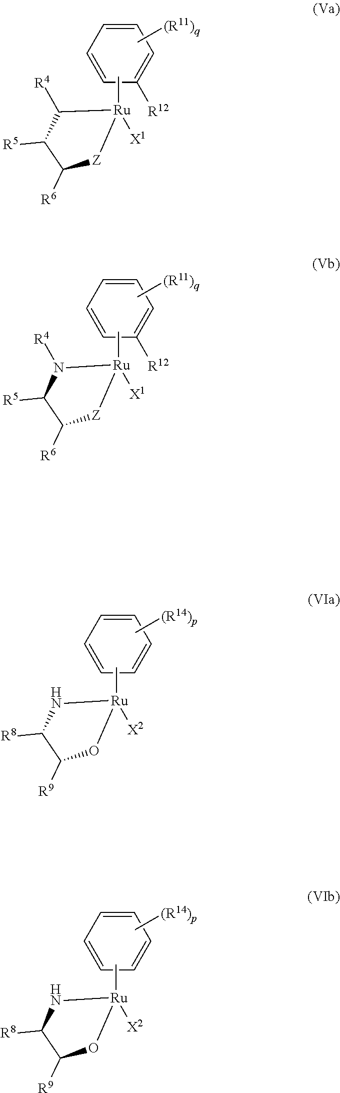

wherein
Z is NR¹³ or O,
R⁴ is phenylsulphonyl, wherein the phenyl is unsubstituted or substituted with one or more substituents independently selected from C₁-C₄-alkyl and halogen, or
R⁴ is 2-pyrrolidinylcarbonyl or 2-piperidinylcarbonyl, preferably (2S)-2-pyrrolidinylcarbonyl,
R⁵ and R⁶ together form a —(CH₂)₃— or —(CH₂)₄— group, or
R⁵ and R⁶ are independently of one another selected from phenyl, which is unsubstituted or substituted with one or more substituents independently selected from C₁-C₄-alkyl,
each R¹¹, if present, is independently selected from C₁-C₄-alkyl,
R¹² is C₁-C₄-alkyl or hydrogen and R¹³ is hydrogen, or
R¹² and R¹³ form together a —(CH₂)₃—, —(CH₂)₄—, —CH₂—O—CH₂—, *—(CH₂)₂—O—CH₂—# or *—(CH₂)—O—(CH₂)₂—# group, where the bond marked with “*” is bonded to the nitrogen and the bond marked with “#” is bonded to the phenyl ring,
q is 0, 1, 2, 3, 4 or 5,
X¹ is chlorine or bromine, or
X¹ BF₄⁻, PF₆⁻ or SbF₆⁻, in which case the Ru—X¹ bond is of a coordinative or ionic nature and the Ru has a positive charge,
R⁸ is C₂-C₆-alkyl and R⁹ is hydrogen, or
R⁸ and R⁹ are independently of one another selected from phenyl, which is unsubstituted or substituted with one or more substituents independently selected from C₁-C₄-alkyl, or
R⁸ and R⁹ form together a group of the formula

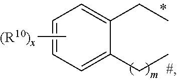

where the bond marked with “*” is connected to the carbon bearing the hydroxyl group and the bond marked with “#” is connected to the carbon bearing the amino group, and wherein
m is 0 or 1,
x is 0, 1 or 2, and
each R¹⁰, if present, is independently selected from C₁-C₄-alkyl.
each R¹⁴, if present, is independently selected from C₁-C₄-alkyl,
p is 0, 1, 2, 3, 4, 5 or 6, and
X² is chlorine or bromine, or
X² is BF₄⁻, PF₆⁻ or SbF₆⁻, in which case the Ru—X² bond is of a coordinative or ionic nature and the Ru has a positive charge.

Depending on whether compound (Ia) or (Ib) is the desired product, a catalyst of the formula (Va) or (VIb) or a catalyst of the formula (Vb) or (VIa) is suitable for use in the process according to the invention. Usually, if a compound of the formula (Ia) is the desired product, a catalyst of the formula (Va) or (VIb), preferably (Va), is suitable for use in the process according to the invention, whereas if a compound of the formula (Ib) is the desired product, a catalyst of the formula (Vb) or (VIa), preferably (Vb), is suitable for use in the process according to the invention.

More preferably, the chiral ruthenium catalyst has the general formula (Va), (Vb), (VIa) or (VIb), where
Z is NR¹³ or O,
R⁴ is phenylsulphonyl, wherein the phenyl is unsubstituted or substituted with one or more substituents independently selected from C₁-C₄-alkyl and halogen, or
R⁴ is 2-pyrrolidinylcarbonyl, preferably (2S)-2-pyrrolidinylcarbonyl,
R⁵ and R⁶ together form a —(CH₂)₄— group, or
R⁵ and R⁶ are unsubstituted phenyl,
each R¹¹, if present, is independently selected from C₁-C₄-alkyl,
R¹² is C₁-C₄-alkyl or hydrogen and
R¹³ is hydrogen, or
R¹² and R¹³ form together a —(CH₂)₃—, —(CH₂)₄—, —CH₂—O—CH₂—, *—(CH₂)₂—O—CH₂—# or *—(CH₂)—O—(CH₂)₂—# group, where the bond marked with “*” is bonded to the nitrogen and the bond marked with “#” is bonded to the phenyl ring,
q is 0, 1, 2, 3, 4 or 5,
X¹ is chlorine or bromine, or
X¹ is BF₄⁻, PF₆⁻ or SbF₆⁻, in which case the Ru—X¹ bond is of a coordinative or ionic nature and the Ru has a positive charge,
R⁸ and R⁹ are unsubstituted phenyl, or
R⁸ and R⁹ form together a group of the formula

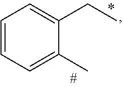

wherein the bond identified by “*” is connected to the carbon bearing the hydroxyl group and the bond identified by “#” is connected to the carbon bearing the amino group,
each R¹⁴, if present, is independently selected from C₁-C₄-alkyl,
p is 0, 1, 2, 3, 4, 5 or 6, and
X² is chlorine or bromine, or
X² is BF₄⁻, PF₆⁻ or SbF₆⁻, in which case the Ru—X² bond is of a coordinative or ionic nature and the Ru has a positive charge.

Particularly preferred are chiral ruthenium catalysts of the general formula (Va) or (Vb), where
Z is NR¹³ or O,
R⁴ is phenylsulphonyl, wherein the phenyl is unsubstituted or substituted with one or more substituents independently selected from C₁-C₄-alkyl and fluorine,
R⁵ and R⁶ together form a —(CH₂)₄— group, or
R⁵ and R⁶ are unsubstituted phenyl, and
each R¹¹, if present, is methyl,
R¹² is C₁-C₄-alkyl, for example methyl or isopropyl, and
R¹³ is hydrogen, or
R¹² and R¹³ form together a —(CH₂)₃—, —(CH₂)₄—, —CH₂—O—CH₂—, *—(CH₂)₂—O—CH₂—# or *—(CH₂)—O—(CH₂)₂—# group, where the bond marked with “*” is bonded to the nitrogen and the bond marked with “#” is bonded to the phenyl ring,
q is 0, 1 or 2,
X¹ is chlorine or bromine, or
X¹ is BF₄⁻, PF₆⁻ or SbF₆⁻, in which case the Ru—X¹ bond is of a coordinative or ionic nature and the Ru has a positive charge.

Chiral ruthenium catalysts of the formulae (Va), (Vb), (VIa) and (VIb) are commercially available or may be prepared by methods known in the art (e.g. R. Hodgkinson et al., Organometallics, 2014, 33, 5517-5524; V. Parekh et al., Catal. Sci. & Technol., 2012, 2, 406-414).

Chiral ruthenium catalysts of the formulae (Va) and (Vb), wherein R¹² is C₁-C₄-alkyl and Z is O or NH, and chiral ruthenium catalysts of the formulae (VIa) and (VIb) may be formed in situ by mixing a dichloro (aromatic ligand)ruthenium (II) dimer precatalyst, such as [RuCl₂(p-cymene)]₂ or [RuCl₂(hexamethylbenzene]₂, or a dibromo (aromatic ligand)ruthenium (II) dimer precatalyst with a chiral ligand of the formula (IIIa′), (IIIb′), (IVa) or (IVb),

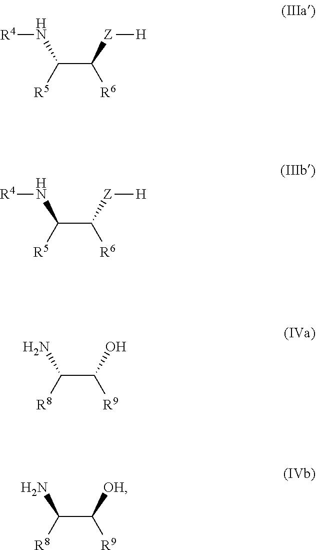

wherein
R⁴, R⁵ and R⁶ are each as defined for the complexes of the formulae (Va) and (Vb),
Z is NH or O,
R⁸ and R⁹ are each as defined for the complexes of the formulae (VIa) and (VIb),
   and wherein the aromatic ligand of the precatalyst is selected from the group consisting of p-cymene and benzene, which is optionally substituted with one or more methyl groups,
   in an organic solvent (e.g. dichloroethane).

Examples of suitable organic solvents are dichloromethane, 1,2-dichloroethane, chlorobenzene, dichlorobenzene, toluene, acetonitrile, dimethylformamide, ethanol, isopropanol, tetrahydrofuran and 2-methyl tetrahydrofurane.

Examples of suitable aromatic ligands are p-cymene and hexamethylbenzene.

The amount of ruthenium catalyst used is preferably within the range of from 0.01 mol % to 10 mol %, more preferably 0.1 mol % to 5 mol %, and most preferably 0.5 mol % to 3 mol %, based on the amount of the compound of the formula (II).

The process according to the invention comprises asymmetric transfer hydrogenation of the compound of the formula (II).

The hydrogen source used is preferably selected from the group consisting of sodium formate, potassium formate, lithium formate, calcium formate, magnesium formate, formic acid/triethylamine, potassium tert-butylate/isopropanol, sodium tert-butylate/isopropanol and lithium tert-butylate/isopropanol, more preferably selected from the group consisting of sodium formate, potassium formate, lithium formate, calcium formate, magnesium formate and formic acid/triethylamine, and most preferably selected from sodium formate and formic acid/triethylamine.

The amount of hydrogen source used is preferably at least 1.0 equivalent, more preferably at least 2.0 equivalents, and most preferably 2.0 to 3.5 equivalents, based on the amount of the compound of the formula (II).

In case of formic acid/triethylamine, the formic acid acts as hydrogen source and the amount of hydrogen source used therefore corresponds to the amount of formic acid used. Preferably, the amount of triethylamine used is within the range of from 0.2 to 1.0 equivalents, based on the amount of the compound of the formula (II).

In case of potassium tert-butylate/isopropanol, sodium tert-butylate/isopropanol and lithium tert-butylate/isopropanol, the isopropanol acts both as a hydrogen source and as a (co-)solvent and the amount of isopropanol used is therefore typically significantly more than the amount of hydrogen source required for the hydrogenation reaction. The amount of tert-butylate used is preferably between 0.2 and 1.0 equivalents, based on the amount of the compound of the formula (II).

Particularly preferably, the hydrogen source used is selected from sodium formate and formic acid/triethylamine and the amount of hydrogen source used is within the range of from 2.0 to 3.5 equivalents, based on the amount of the compound of the formula (II).

The transfer hydrogenation is preferably conducted at a temperature within the range of from 10° C. to 100° C., more preferably 20° C. to 80° C., and in particular 25° C. to 50° C.

The reaction time is not critical and may, according to the batch size, be selected within a relatively wide range. Typical reaction times are between 30 min and 24 h.

According to the invention, the asymmetric transfer hydrogenation of the compound of the formula (II) is conducted in the presence of a polar solvent.

Suitable polar solvents are selected from the group consisting of dichloromethane, methanol, ethanol, isopropanol, n-butanol, tetrahydrofuran, 2-methyl-tetrahydrofuran, dimethylformamide, acetonitrile, methanol/water, ethanol/water, isopropanol/water, n-butanol/water, tetrahydrofuran/water, 2-methyl-tetrahydrofuran/water, dimethylformamide/water, acetonitrile/water, and mixtures thereof.

Preferred polar solvents are selected from the group consisting of ethanol, isopropanol, 2-methyl-tetrahydrofuran, dimethylformamide, acetonitrile, ethanol/water, isopropanol/water, 2-methyl-tetrahydrofuran/water, dimethylformamide/water, acetonitrile/water, and mixtures thereof.

Particularly preferred are ethanol, isopropanol/water, dimethylformamide/water, acetonitrile/water; 2-methyl-tetrahydrofuran and 2-methyl-tetrahydrofuran/water.

If the transfer hydrogenation is conducted in presence of water, the work-up and isolation of the compound of formula (Ia) or (Ib) can be effected by the following steps: (i) separating the aqueous phase from the organic phase, (ii) extracting the aqueous phase one or more times with a suitable organic solvent (e.g. heptane, toluene or xylene), (iii) washing the combined organic phases with water, brine and/or aqueous sodium bicarbonate solution, (iv) drying the obtained organic phase by treatment with magnesium sulfate or via azeotropic distillation and (v) removing (part of) the organic solvent by distillation. The obtained product may be purified by crystallization from heptane.

The compounds of formula (Ia) or (Ib) can be prepared in high enantioselectivity by means of the asymmetric transfer hydrogenation according to the invention. The compound of formula (Ia) or (Ib) obtained by the process according to the invention can be purified by forming a crystalline addition salt with camphor sulfonic acid. This can increase the chemical purity of the desired product to >99% w/w.

The process according to the invention comprises asymmetric transfer hydrogenation of the compound of the formula (II).

Ketones of the formula (II) may be obtained from a racemic mixture of the compounds of formulae (Ia) and (Ib)

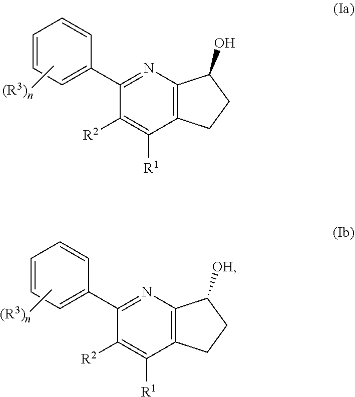

wherein the substituents IV, R², R³ and the integer n are each as defined for the compound of the formula (II),
by oxidation using TEMPO, a TEMPO derivative or a TEMPO analogue, a hypochlorite salt and optionally a bromide salt.

Analogously, ketones of the formula (II′) may be obtained from a racemic mixture of the compounds of the formulae (Ia′) and (Ib′)

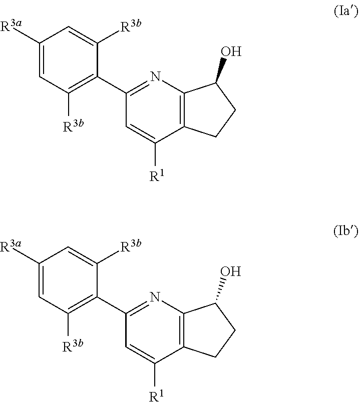

wherein the substituents R¹, R^{3a} and R^{3b} are each as defined for the compound of the formula (II′),
by oxidation using TEMPO, a TEMPO derivative or a TEMPO analogue, a hypochlorite salt and optionally a bromide salt.

It has been found that the ketone of formula (II) may be obtained from a racemic mixture of the compounds of formulae (Ia) and (Ib) by a TEMPO-mediated oxidation using catalytic amounts of TEMPO, a TEMPO derivative or a TEMPO analogue, a hypochlorite salt as oxidation agent and optionally bromide as co-oxidation agent. It has been found that this reaction works well with compounds of the formula (Ia) and (Ib) having a pyridine functionality, which is surprising in light of previous results disclosed in M. Shibuya, M. Tomizawa, I. Suzuki, Y. Iwabuchi, J. Am. Chem. Soc., 2006, 128, 8412-8413. Shibuya et al. teach that nitrosyl radicals, such as TEMPO and 1-Me-AZADO, do not efficiently oxidize substrates containing a basic nitrogen. Moreover, the reaction also works well using catalytic amounds of readily available TEMPO, which is surprising because Shibuya et al. teach to use 1-Me-AZADO (2-aza-1-methyladamantane N-oxyl) instead of TEMPO for the oxidation of secondary alcohols.

Examples of suitable TEMPO derivatives and TEMPO analogues are 4-hydroxy-TEMPO, 4-methoxy-TEMPO, 4-oxo-TEMPO, 2-azaadamantane N-oxyl and 2-aza-1-methyladamantane N-oxyl.

The TEMPO, TEMPO derivative or TEMPO analogue may be used as such or in immobilized form. Suitable examples of immobilized TEMPO are silica-supported TEMPO and polystyrene-supported TEMPO.

The amount of TEMPO, TEMPO derivative or TEMPO analogue used is preferably within the range of from 0.5 mol % to 20 mol %, more preferably 1 mol % to 10 mol %, most preferably 3 mol % to 7.5 mol %, based on the total amount of the compounds of formulae (Ia) and (Ib), preferably (Ia′) and (Ib′).

Suitable hypochlorite salts are sodium hypochlorite, potassium hypochlorite and magnesium hypochlorite. Preferably, the hypochlorite salt used in the TEMPO-mediated oxidation is selected from sodium hypochlorite and potassium hypochlorite. Sodium hypochlorite is particularly preferred.

The amount of hypochlorite salt used is preferably within the range of from 1 to 5 equivalents, more preferably 1.1 to 2.0 equivalents, most preferably 1.2 to 1.5 equivalents, based on the total amount of the compounds of the formulae (Ia) and (Ib), preferably (Ia′) and (Ib′).

Suitable bromide salts are potassium bromide, sodium bromide and tetrabutylammonium bromide, and mixtures thereof.

The amount of bromide salt used is preferably within the range of from 0.5 mol % to 20 mol %, more preferably 5 mol % to 15 mol %, based on the total amount of the compounds of the formulae (Ia) and (Ib), preferably (Ia′) and (Ib′).

The TEMPO-mediated oxidation is preferably conducted under basic two-phase conditions in presence of water, an organic solvent and a phase-transfer catalyst, such as tetrabutylammoniumbromide (TBAB).

Suitable organic solvents are selected from the group consisting of dichloromethane, 1,2-dichloroethane, chlorobenzene, dichlorobenzene, toluene, acetonitrile, ethyl acetate, n-propyl acetate, n-butyl acetate and similar solvents that are inert towards oxidation by hypochlorite reagents.

For example, the TEMPO-mediated oxidation may be conducted under basic two-phase conditions using a mixture of an aqueous sodium hypochlorite solution and a saturated aqueous solution of sodium bicarbonate as aqueous phase, dichloromethane as organic solvent and tetrabutylammoniumbromide (TBAB) as phase-transfer catalyst.

The TEMPO-mediated oxidation is preferably conducted at a temperature within the range of from −20° C. to +25° C., preferably −5° C. to +5° C.

The reaction time is not critical and may, according to the batch size, be selected within a relatively wide range. Typical reaction times are between 5 min and 3 h.

The work-up and isolation of the ketone of formula (II) or (II′) may be effected by the following steps: (i) separating the aqueous phase from the organic phase, (ii) extracting the aqueous phase one or more times with a suitable organic solvent (e.g. heptane), (iii) washing the combined organic phases with water or brine, (iv) drying the obtained organic phase by treatment with magnesium sulfate and (v) removing the organic solvent by distillation. The obtained product of formula (II) or (II′) may be purified by crystallization from heptane.

### Abbreviations and Acronyms

| | |
|---|---|
| % a/a | area percentage |
| DCE | 1,2-dichloroethane |
| DMF | dimethylformamide |
| ee | enantiomeric excess |
| Et | ethyl |
| EtOH | ethanol |
| HPLC | high performance liquid chromatography |
| Ph | phenyl |
| ⁱPrOH | isopropanol |
| qNMR | quantitative NMR |
| Me | methyl |
| MeCN | acetonitrile |
| MeTHF | 2-methyl tetrahydrofurane |
| NEt₃ | triethylamine |
| NMR | nuclear magnetic resonance |
| TEMPO | 2,2,6,6-tetramethylpiperidinyloxyl |
| TMB | trimethoxybenzene |
| Ts | tosyl (p-toluenesulfonyl) |

### EXAMPLES

### Preparation of the Starting Material (II′-1) by TEMPO-Mediated Oxidation of (Ia′-1)/(Ib′-1)

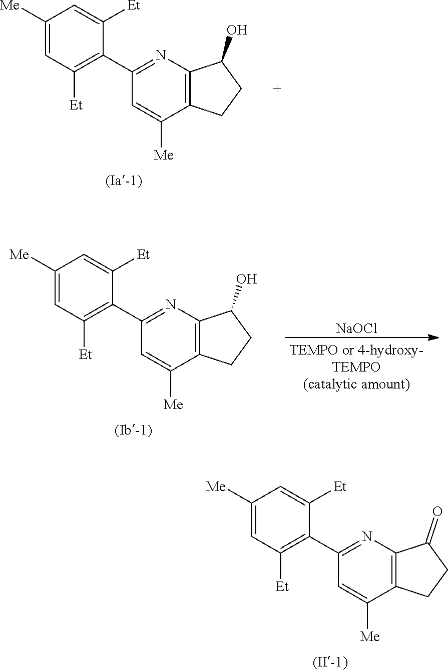 [SMILES: CCc1cc(C)cc(CC)c1-c1cc(C)c2c(n1)[C@@H](O)CC2 ; CCc1cc(C)cc(CC)c1-c1cc(C)c2c(n1)[C@H](O)CC2 ; "NaOCl" ; CCc1cc(C)cc(CC)c1-c1cc(C)c2c(n1)C(=O)CC2]

### Example 1

A racemic mixture of compounds (Ia′-1) and (Ib′-1) (93.3% w/w, 1421.5 g, 4489 mmol), TEMPO (35 g, 224 mmol), potassium bromide (53 g, 449 mmol), tetrabutylammonium bromide (72 g, 224 mmol), dichloromethane (6.8 L) and saturated sodium bicarbonate solution (made with 4.5 L of water) were placed in a reactor. The beige colored mixture was cooled down to 0° C. and a mixture of sodium hypochlorite solution (13.4% w/w, total amount needed: 3530 g, 5454 mmol, 1.215 equiv) and saturated sodium bicarbonate solution (total amount needed: 3.81 kg) was added at 0° C. (±4° C.) under temperature control until in-process control (HPLC@220 nm) showed complete conversion of starting material. The reaction mixture was transferred into a stirring vessel and was diluted with water (2.8 L). The aqueous phase was separated and re-extracted with dichloromethane (5.6 L). The combined organic layers were washed with water (5.6 L) and filtered through a sodium sulfate plug (1 kg), which was rinsed with dichloromethane (2.8 L). 10 L of solvent were distilled off (40° C.) and 6 L of heptane were added. Again 4.5 L of solvent were distilled off and replaced by heptane. 1 L of heptane was distilled off and the solution was seeded by adding 2 g of compound (II′-1) to initiate crystallization. The suspension was concentrated at 45° C. to a total mass of 8 kg, cooled down and rotated at 0-5° C. for 3 hours. The solid was filtered off and washed with cold heptane (5 L, 0-5° C.). The solid was dried under vacuum at 40-45° C.

Mass: 1273 g (97% of theory); Appearance: beige solid; HPLC (220 nm): ≥99% area; Assay (1H-NMR, DMSO-d6, TMB as Standard): 96%. Yield (mass yield x assay): 93% of compound (II′-1).

### Example 2

A racemic mixture of compounds (Ia′-1) and (Ib′-1) (0.13 g, 0.44 mmol), 4-hydroxy-TEMPO (3.8 mg, 0.022 mmol), potassium bromide (15 mg, 0.044 mol), tetrabutylammonium bromide (7.1 mg, 0.022 mmol), dichloromethane (2.6 mL) and saturated sodium bicarbonate solution (1.3 mL) were placed in a vial under inert atmosphere (N₂). The beige colored mixture was cooled down to 0° C. and a mixture of sodium hypochlorite solution (10-14% w/w, 0.7 mL) and saturated sodium bicarbonate solution (0.9 mL) was added dropwise at 0° C. (±4° C.) during 5 min. After 20 min stirring at this temperature, in-process control (HPLC@220 nm) showed complete conversion of starting material and 82.9% a/a of compound (II′-1).

### Example 3

A racemic mixture of compounds (Ia′-1) and (Ib′-1) (0.13 g, 0.44 mmol), silica-supported TEMPO (0.35 mmol TEMPO per gramm of material, 63 mg, 0.022 mmol), potassium bromide (15 mg, 0.044 mol), tetrabutylammonium bromide (7.1 mg, 0.022 mmol), dichloromethane (2.6 mL) and saturated sodium bicarbonate solution (1.3 mL) were placed in a vial under inert atmosphere (N₂). The beige colored mixture was cooled down to 0° C. and a mixture of sodium hypochlorite solution (10-14% w/w, 0.7 mL) and saturated sodium bicarbonate solution (0.9 mL) was added dropwise at 0° C. (±4° C.) during 5 min. After 20 min stirring at this temperature, in-process control (HPLC@220 nm) showed complete conversion of starting material and 96.6% a/a of compound (II′-1).

### Example 4

A racemic mixture of compounds (Ia′-1) and (Ib′-1) (0.13 g, 0.44 mmol), polystyrene-supported TEMPO (1 mmol TEMPO per gramm of material, 22 mg, 0.022 mmol), potassium bromide (15 mg, 0.044 mol), tetrabutylammonium bromide (7.1 mg, 0.022 mmol), dichloromethane (2.6 mL) and saturated sodium bicarbonate solution (1.3 mL) were placed in a vial under inert atmosphere (N₂). The beige colored mixture was cooled down to 0° C. and a mixture of sodium hypochlorite solution (10-14% w/w, 0.7 mL) and saturated sodium bicarbonate solution (0.9 mL) was added dropwise at 0° C. (±4° C.) during 5 min. After 20 min stirring at this temperature, in-process control (HPLC@220 nm) showed complete conversion of starting material and 90.4% a/a of compound (II′-1).

### Asymmetric Transferhydrogenation

Reactions were performed in glass vessels of appropriate dimensions. Unless stated otherwise, reaction mixtures were analyzed without workup via HPLC (Chiralpak IC column, heptane/ethanol gradient (with 0.02% of diethylamine as stabilizing additive), 1 mL/min).

### Preparation of Chiral Ruthenium Catalysts

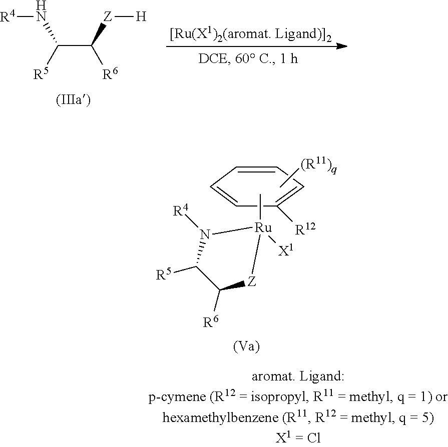

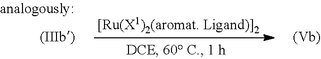

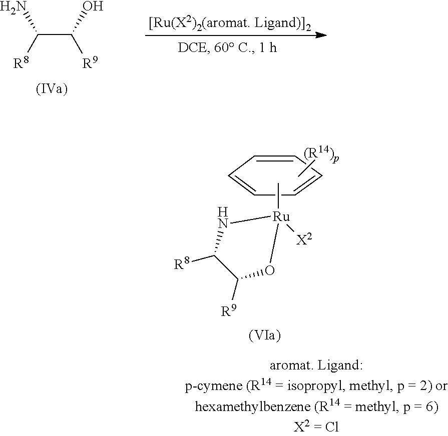

The catalysts used in examples 5-14 were preformed prior to reaction by dissolving a ruthenium (II) catalyst precursor ([RuCl₂(p-cymene)]₂ or [RuCl₂(hexamethylbenzene]₂, 1.0 equiv.) in DCE at 60° C., adding the ligand given in table 1 (1.2 equiv.) and stirring of the solution for 1 h at 60° C., followed by evaporation of DCE.

The following catalysts are commercially available and were used as purchased in examples 15-34:

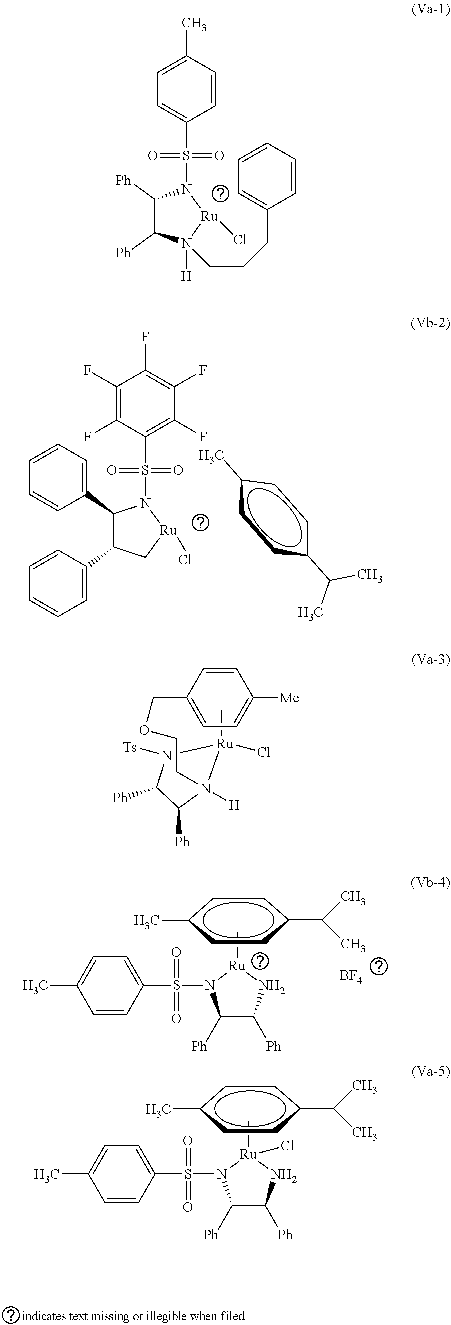

### Transfer Hydrogenation Reaction

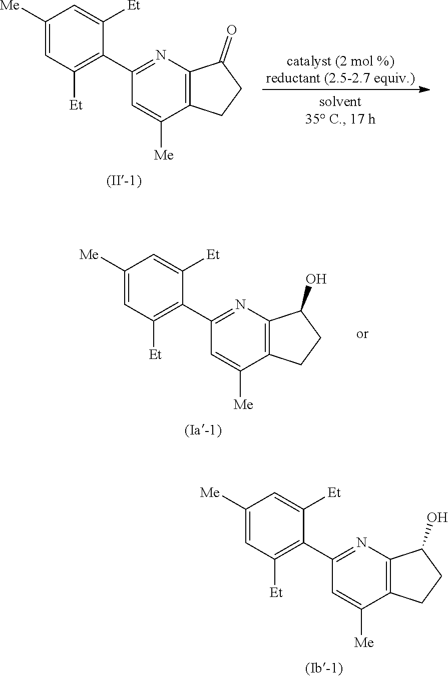 [SMILES: CCc1cc(C)cc(CC)c1-c1cc(C)c2c(n1)C(=O)CC2 ; "catalyst (2 mol %)reductant (2.5-2.7 equiv.)" ; CCc1cc(C)cc(CC)c1-c1cc(C)c2c(n1)[C@@H](O)CC2 ; CCc1cc(C)cc(CC)c1-c1cc(C)c2c(n1)[C@H](O)CC2]

Under an inert gas atmosphere, one well of a 96 well-plate autoclave was filled with 9.7 mg of ketone starting material (II′-1) (33 μmol, 1 equiv), reductant (see table 1; NaCO₂H: 2.5 equiv.; HCO₂H/NEt₃: 2.7 equiv./0.6 equiv., respectively), 0.66 μmol of catalyst (2 mol %, see table 1) in the respective solvent mixture (see table 1, starting material concentration is 0.13 M). The autoclave was closed and heated to 35° C. and the reaction mixture was shaken at that temperature for 17 h. Chromatographic analysis of the cooled and de-pressurized reaction mixture showed the % a/a HPLC conversion rates of starting material (II′-1) to the reduced alcohol product (Ia′-1) or (Ib′-1). The % a/a HPLC conversion rates and enantioselectivities are depicted in table 1 below.

TABLE 1

| | Catalyst | | | | Product | Conversion | |
|---|---|---|---|---|---|---|---|
| | Precursor¹⁾/ | | | | (major | (% a/a | % |
| Ex. | Catalyst²⁾ | Ligand | Solvent | Reductant | enantiomer) | HPLC) | ee |
| 5 | [RuCl₂(p-cymene)]₂ | 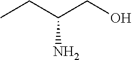 | iPrOH; H₂O | HCO₂Na | (Ib′-1) | 79.9 | 56.4 |
| 6 | [RuCl₂(p-cymene)]₂ | 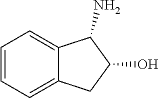 [SMILES: N[C@H]1c2ccccc2C[C@H]1O] | DMF; H₂O | HCO₂Na | (Ib′-1) | 43.9 | 80.3 |
| 7 | [RuCl₂(p-cymene)]₂ | 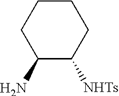 | ⁱPrOH; H₂O | HCO₂Na | (Ia′-1) | 100 | 92.3 |
| 8 | [RuCl₂(hexa- methylbenzene)]₂ | 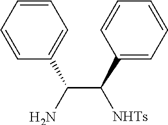 | MeTHF; H₂O | HCO₂Na | (Ib′-1) | 100 | 97.9 |
| 9 | [RuCl₂(p-cymene)]₂ | 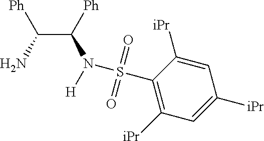 [SMILES: CC(C)c1cc(C(C)C)c(S(=O)(=O)N[C@H](c2ccccc2)[C@H](N)c2ccccc2)c(C(C)C)c1] | MeCN; H₂O | HCO₂Na | (Ib′-1) | 100 | 96.1 |
| 10 | [RuCl₂(p-cymene)]₂ | 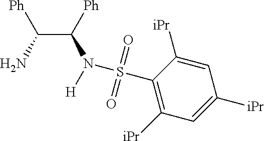 [SMILES: CC(C)c1cc(C(C)C)c(S(=O)(=O)N[C@H](c2ccccc2)[C@H](N)c2ccccc2)c(C(C)C)c1] | DMF | HCO₂H; NEt₃ | (Ib′-1) | 86.9 | 89.1 |
| 11 | [RuCl₂(p-cymene)]₂ | 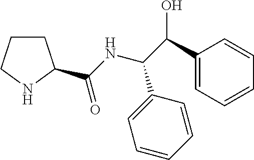 [SMILES: O=C(N[C@@H](c1ccccc1)[C@@H](O)c1ccccc1)[C@@H]1CCCN1] | DMF; H₂O | HCO₂Na | (Ib′-1) | 100 | 96.8 |
| 12 | [RuCl₂(p-cymene)]₂ | 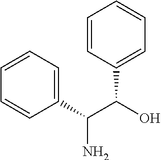 [SMILES: N[C@H](c1ccccc1)[C@@H](O)c1ccccc1] | MeTHF; H₂O | HCO₂Na | (Ia′-1) | 97.2 | 84 |
| 13 | [RuCl₂(hexa- methylbenzene)]₂ | 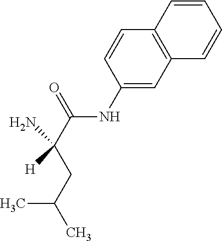 [SMILES: CC(C)C[C@H](N)C(=O)Nc1ccc2ccccc2c1] | MeCN; H₂O | HCO₂Na | (Ib′-1) | 98.4 | 30.4 |
| 14 | [RuCl₂(p-cymene)]₂ | 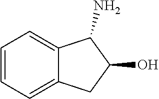 [SMILES: N[C@H]1c2ccccc2C[C@@H]1O] | EtOH; H₂O | HCO₂Na | (Ib′-1) | 21.8 | 16.7 |
| 15 | (Va-1) | — | EtOH | HCO₂H; | (Ia′-1) | 100 | 97.5 |
| | | | | NEt₃ | | | |
| 16 | (Va-1) | — | DMF; | HCO₂Na | (Ia′-1) | 98.9 | 95.9 |
| | | | H₂O | | | | |
| 17 | (Va-1) | — | MeTHF | HCO₂H; | (Ia′-1) | 83.0 | 93.6 |
| | | | | NEt₃ | | | |
| 18 | (Va-1) | — | MeCN; | HCO₂Na | (Ia′-1) | 99.1 | 97.3 |
| | | | H₂O | | | | |
| 19 | (Vb-2) | — | EtOH | HCO₂H; | (Ib′-1) | 100 | 97.1 |
| | | | | NEt₃ | | | |
| 20 | (Vb-2) | — | DMF; | HCO₂Na | (Ib′-1) | 100 | 94.5 |
| | | | H₂O | | | | |
| 21 | (Vb-2) | — | MeTHF | HCO₂H; | (Ib′-1) | 100 | 95.5 |
| | | | | NEt₃ | | | |
| 22 | (Vb-2) | — | MeCN; | HCO₂Na | (Ib′-1) | 100 | 96.9 |
| | | | H₂O | | | | |
| 23 | (Va-3) | — | EtOH | HCO₂H; | (Ia′-1) | 100 | 92.1 |
| | | | | NEt₃ | | | |
| 24 | (Va-3) | — | DMF; | HCO₂Na | (Ia′-1) | 98.5 | 98.5 |
| | | | H₂O | | | | |
| 25 | (Va-3) | — | MeTHF | HCO₂H; | (Ia′-1) | 96.6 | 91.6 |
| | | | | NEt₃ | | | |
| 26 | (Va-3) | — | MeCN; | HCO₂Na | (Ia′-1) | 100 | 98.7 |
| | | | H₂O | | | | |
| 27 | (Vb-4) | — | EtOH | HCO₂H; | (Ib′-1) | 100 | 96.2 |
| | | | | NEt₃ | | | |
| 28 | (Vb-4) | — | MeTHF | HCO₂H; | (Ib′-1) | 100 | 96.6 |
| | | | | NEt₃ | | | |
| 29 | (Vb-4) | — | DMF; | HCO₂Na | (Ib′-1) | 98.2 | 96.3 |
| | | | H₂O | | | | |
| 30 | (Va-5) | — | EtOH | HCO₂H; | (Ia′-1) | 100 | 94.1 |
| | | | | NEt₃ | | | |
| 31 | (Va-5) | — | MeTHF | HCO₂H; | (Ia′-1) | 100 | 91.9 |
| | | | | NEt₃ | | | |
| 32 | (Va-5) | — | DMF; | HCO₂Na | (Ia′-1) | 98.9 | 96.7 |
| | | | H₂O | | | | |

¹⁾: The catalysts used in examples 5-12 are complexes of the formula (Va), (Vb) or (VIa). These catalysts were preformed prior to the reaction from the catalyst precursors and ligands given in the table according to the method described above. The catalysts used in examples 13 and 14 were prepared accordingly.
2): The catalysts used in examples 15-32 are commercially available and were employed as purchased.

### Example 33

All solvents and solutions for the reaction and work-up procedure were degassed with argon before use. Ketone starting material (II′-1) (9.4 g, 32 mmol) and ethanol (70 ml) were placed in a 50 ml three necked round bottom flask under argon atmosphere. Argon was bubbled through the suspension for 15 minutes before catalyst (Va-3) (2 mol %, 379 mg, 0.64 mmol) was added. A solution of sodium formate (24 g, 352 mmol) in water (94 ml) was added. The reaction was stirred at 35° C. (bath temperature) overnight (16 h). In a separation funnel, the oily upper layer was separated and the aqueous phase was extracted with heptane (50 ml). The combined upper layers were diluted with heptane (25 ml) and washed with water (50 ml). The separated aqueous phase was re-extracted with heptane (40 ml). The combined organic phases were washed with water (50 ml) and brine (aqueous, 30%, 30 ml).

### Purification by Silica Plug Filtration

A column was filled with silica gel 60 (Fluka 89943, 50 g) as slurry in heptane. The organic layer from the extraction was directly applied on the column and eluted with a gradient from heptane (100%) to heptane/MeTHF 3/1 (v/v). Product fractions were evaporated in vacuum yielding 9.3 g of beige/brown solid (Assay: 96% w/w, 94% yield, 97% ee).

### Example 34

The reaction was performed under inert gas atmosphere. All solvents and solutions for the reaction and work-up procedure were degassed with argon prior to use. Compound (II′-1) (1230 g, 4025 mmol) and catalyst (Va-3) (54 g, 80 mmol) were placed in 20-L round bottom flask under inert gas atmosphere (Argon). Acetonitrile (4 L) was added and the mixture was mixed (30° C.) to get a brownish to red solution (Solution 1). Sodium formate (1369 g, 20.1 mol) was dissolved in degassed water (7 L). The solution was three times evacuated and flushed with argon (Solution 2). Solution 1 was placed in a reactor (flask rinsed with 0.5 L of acetonitrile) followed by Solution 2 (flask rinsed with 1 L of water).

The mixture was heated up to 35° C. within about 45 minutes and stirred at this temperature for one hour. Process control showed full conversion of the starting material (II′-1). The reaction mixture was cooled down to 25° C. and transferred into a separation vessel and the phases were separated. The aqueous layer was re-extracted with Heptane (3.7 L). The mixed organic phases (biphasic mixture) was washed with 2×1.85 L half saturated aqueous sodium bicarbonate, followed by 1.85 L of saturated aqueous sodium bicarbonate solution. The organic layer was filtered over a sodium sulfate plug (800 g) and rinsed with heptane (2×1 L). The solvent was evaporated under vacuum (45° C.) giving 1260 g of a brownish to violet resin.

Analytics: HPLC achiral (220 nm): 97.6% area, HPLC chiral (220 nm): ee 99.7%. The chemical yield was determined after purification via salt-formation and freebasing (cf. example 35).

### Camphersulfonic Acid Salt Formation

### Example 35

Crude (Ia′-1) (from example 34, 1321 g) was dissolved in MeTHF (7 L) at 50° C. A solution of (1S)-(+)-10-camphor sulfonic acid (981 g, 4221 mmol) in MeTHF (4 L) was added continuously at 50° C. within 20 minutes; the solution was seeded while adding. After complete addition, the formed suspension was stirred for additional 30 minutes at 50° C. and then cooled down to 20° C. within 1 hour. The solid was filtered off, washed with MeTHF (2×1 L) and dried under vacuum at 45° C.

Yield: 1947 g (87% of theory) white solid, HPLC (220 nm): ≥99% area

1945 g of this material was dissolved in MeTHF (13 L) and water (5 L). 1.65 L of aqueous, saturated Na₂CO₃-solution was added to increase to pH to 10). The phases were separated, and the organic layer was washed with water (3.3 L) and brine (30%, 1.6 L). The organic layer was filtered over a sodium sulfate plug (1 kg) and rinsed with MeTHF (1.5 L). The solvent was evaporated in vacuum (45° C.). The residue was co-evaporated with heptane (3×1.3 L).

Yield: 1087 g (beige solid). Analytics: 99.7% qNMR (DMSO-d6, internal standard: trimethoxybenzene); 99.7% ee (Chiralpak IC column, heptane/ethanol gradient (with 0.02% of diethylamine as stabilizing additive), 1 mL/min, 220 nm). Purity corrected yield: 87% over 3 steps (transferhydrogenation (example 34), salt-formation, freebasing).

### Example 36

The reaction was performed under inert gas atmosphere. All solvents and solutions for the reaction and work-up procedure were degassed with nitrogen prior to use. Compound (II′-1) (177 g, 589 mmol) and catalyst (Va-3) (1.92 g, 2.95 mmol, 0.5 mol %) were placed in a round bottom flask under inert gas atmosphere (Argon). Acetonitrile (646 mL) was added and the mixture was mixed to get a brownish to red solution (Solution 1). Sodium formate (200.4 g, 2947 mmol) was dissolved in degassed water (1.15 L). The solution was further degassed by bubbling through nitrogen for 1 h (Solution 2). Solution 2 was placed in a reactor followed by Solution 1.

The mixture was heated up to 35° C. within about 35 minutes and stirred at this temperature overnight. Process control showed full conversion of the starting material (II′-1). The reaction mixture was cooled down to 25° C. and transferred into a separation vessel and the phases were separated. From the organic layer, most of the acetonitrile is removed under reduced pressure (150-100 mbar) and a jacket temperature of 40° C. The aqueous layer was re-extracted with xylene (233 g). The xylene layer is added to the distillation sump of the acetonitrile layer. Again, vacuum is applied (100 mbar, 50° C. jacket temperature), removing residues of acetonitrile, water, and part of the xylene (distillate amount: 233 g). A solution of (1S)-(+)-10-camphor sulfonic acid (136 g, 585 mmol) in MeTHF (420 g) was added continuously at 50° C. within 30 minutes. The mixture is kept at that temperature for 50 min, cooled to 10° C. within 2 h and then kept at 10° C. for a further 2 h. The mixture is filtered and washed twice with 226 g of MeTHF each. The filter cake is dried under vacuum at 30° C. yielding 288 g of Camphersulfonic acid salt in ≥99% a/a purity (92% yield (uncorrected) over two steps) and with an ee of 99.4%.

### Example 37 (Freebasing)

124 g of Camphersulfonic acid salt was dissolved in toluene (428 g), MeTHF, (48 g) and water (425 g) together with 2 g of sodium hydrogen carbonate. 49.3 g of 20% w/w aqueous sodium hydroxide solution were added dropwise to increase the pH to 10. The mixture was heated to 40-50° C., filtered clear, and the phases were separated. The organic layer was washed at 40-50° C. with a 5% w/w aqueous solution of sodium hydrogen carbonate (210 mL) and then evaporated to dryness to yield 70.5 g of Ia′-1 as a beige solid (99% yield, purity: 98% w/w.; 99.4% ee (Chiralpak IC column, heptane/ethanol gradient (with 0.02% of diethylamine as stabilizing additive), 1 mL/min, 220 nm). Alternatively, the distillation can be stopped before completion to obtain Ia′-1 as a 50% w/w solution in toluene.

### Example 38

The reaction was performed under inert gas atmosphere. All solvents and solutions for the reaction and work-up procedure were degassed with nitrogen prior to use. Compound (II′-1) (93.9% purity, 78.5 g, 251 mmol) and catalyst (Va-3) (0.817 g, 1.25 mmol, 0.5 mol %) were placed in a round bottom flask under inert gas atmosphere (nitrogen). Acetonitrile (302 mL) was added and the mixture was mixed for 3 h under a constant flow of nitrogen to get a brownish to red solution (Solution 1). Sodium formate (85.4 g, 1256 mmol) was dissolved in degassed water (537 mL). The solution was further degassed by bubbling through nitrogen (Solution 2). Solution 2 was placed in a reactor followed by Solution 1.

The mixture was heated up to 35° C. within about 35 minutes and stirred for 6 h. Process control showed full conversion of the starting material (II′-1). The reaction mixture was cooled down to 25° C. and transferred into a separation vessel and the phases were separated. From the organic layer, most of the acetonitrile is removed under reduced pressure (150-100 mbar) and a jacket temperature of 40° C. The aqueous layer was re-extracted with xylene (162 g). The xylene layer is added to the distillation sump of the acetonitrile layer. Again, vacuum is applied (100-40 mbar, 50° C. jacket temperature), removing residues of acetonitrile, water, and part of the xylene (distillate amount: 139 g). At 50° C., 1 g of seed crystals are added. Then a solution of (1S)-(+)-10-camphor sulfonic acid (58.3 g, 251 mmol) in MeTHF (241 g) is added continuously within 30 minutes at 50° C. under fast stirring. The mixture is kept at that temperature for 30 min, cooled to 10° C. within 2 h and then kept at 10° C. overnight. The mixture is filtered and washed twice with 100 g of MTBE each. The filter cake is dried under vacuum at 30° C. yielding 122 g of Camphersulfonic acid salt (89% yield over two steps) in 97.1% w/w assay purity and with an ee of 99.4%.

1. A process for preparing a compound of formula (Ia) or (Ib), 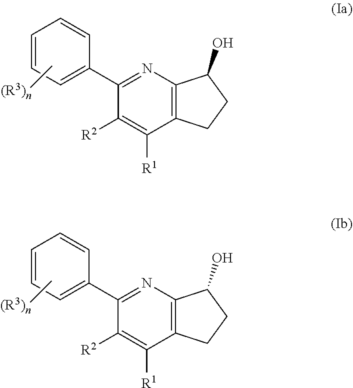
where
R¹ and R² are independently from one another selected from the group consisting of hydrogen and C₁-C₄-alkyl,
each R³, if present, is independently selected from C₁-C₄-alkyl, and
n is 0, 1, 2 or 3,
comprising asymmetric transfer hydrogenation of a ketone of formula (II) 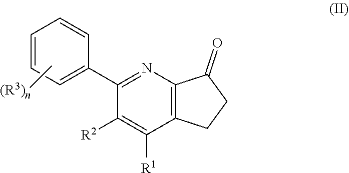
in which the substituents R¹, R², R³ and the integer n are as defined for the compound of formula (Ia) or (Ib),
in presence of a chiral ruthenium catalyst and a polar solvent, wherein the ruthenium catalyst comprises a chiral amino alcohol ligand or a chiral diamine ligand.

2. The process according to claim 1, wherein the chiral ruthenium catalyst comprises a chiral ligand of formula (IIIa), (IIIb), (IVa) or (IVb) 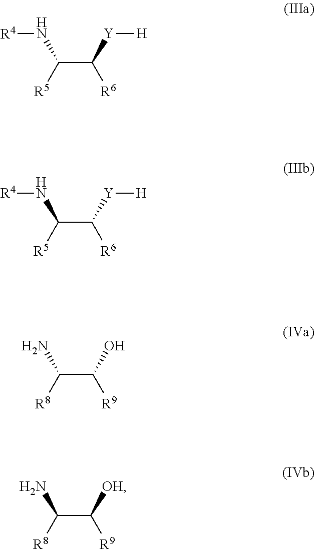
where
Y is NR⁷ or O,
R⁴ is phenylsulphonyl, wherein the phenyl is unsubstituted or substituted with one or more substituents independently selected from C₁-C₄-alkyl and halogen, or
R⁴ is 2-pyrrolidinylcarbonyl or 2-piperidinylcarbonyl,
R⁵ and R⁶ together form a —(CH₂)₃— or —(CH₂)₄— group, or R⁵ and R⁶ are independently of one another selected from phenyl, which is unsubstituted or substituted with one or more substituents independently selected from C₁-C₄-alkyl,
R⁷ is hydrogen, phenyl-(CH₂)₃—, phenyl-(CH₂)₄—, benzyloxymethyl, benzyloxyethyl or phenyl-(CH₂)₂—O—CH₂—, wherein the phenyl and benzyl groups are optionally substituted with one or more substituents independently selected from C₁-C₄-alkyl,
R⁸ is C₂-C₆-alkyl and R⁹ is hydrogen, or
R⁸ and R⁹ are independently of one another selected from phenyl, which is unsubstituted or substituted with one or more substituents independently selected from C₁-C₄-alkyl, or
R⁸ and R⁹ form together a group of formula 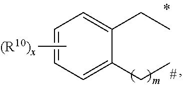
wherein the bond marked with “*” is connected to the carbon bearing the hydroxyl group and the bond marked with “#” is connected to the carbon bearing the amino group, and wherein
m is 0 or 1,
x is 0, 1 or 2, and
each R¹⁰, if present, is independently selected from C₁-C₄-alkyl.

3. The process according to claim 1, wherein the chiral ruthenium catalyst has formula (Va), (Vb), (VIa) or (VIb): 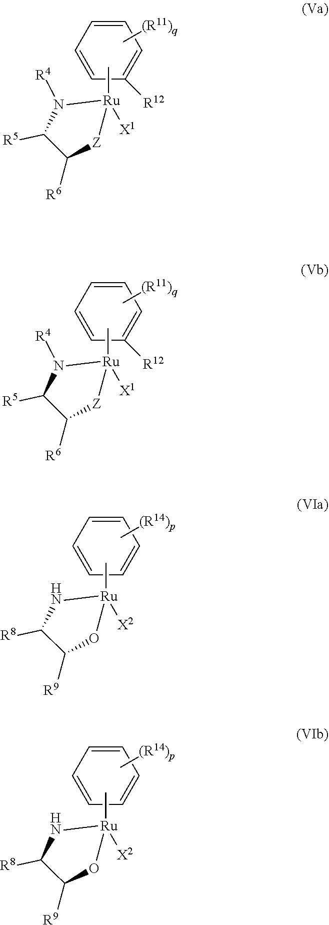
where
Z is NR¹³ or O,
R⁴ is phenylsulphonyl, wherein the phenyl is unsubstituted or substituted with one or more substituents independently selected from C₁-C₄-alkyl and halogen, or
R⁴ is 2-pyrrolidinylcarbonyl,
R⁵ and R⁶ together form a —(CH₂)₄— group, or
R⁵ and R⁶ are unsubstituted phenyl, each R¹¹, if present, is independently selected from C₁-C₄-alkyl,
R¹² is C₁-C₄-alkyl or hydrogen and
R¹³ is hydrogen, or
R¹² and R¹³ form together a —(CH₂)₃—, —(CH₂)₄—, —CH₂—O—CH₂—, *—(CH₂)₂—O—CH₂—# or
*—(CH₂)—O—(CH₂)₂—# group, where the bond marked with “*” is bonded to the nitrogen and the bond marked with “#” is bonded to the phenyl ring,
q is 0, 1, 2, 3, 4 or 5,
X¹ is chlorine or bromine, or
X¹ is BF₄⁻, PF₆⁻ or SbF₆⁻, in which case the Ru—X¹ bond is of a coordinative or ionic nature and the Ru has a positive charge,
R⁸ and R⁹ are unsubstituted phenyl, or
R⁸ and R⁹ form together a group of formula 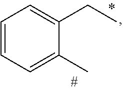
wherein the bond identified by “*” is connected to the carbon bearing the hydroxyl group and the bond identified by “#” is connected to the carbon bearing the amino group,
each R¹⁴, if present, is independently selected from C₁-C₄-alkyl,
p is 0, 1, 2, 3, 4, 5 or 6, and
X² is chlorine or bromine, or
X² is BF₄⁻, PF₆⁻ or SbF₆⁻, in which case the Ru—X² bond is of a coordinative or ionic nature and the Ru has a positive charge.

4. The process according to claim 2, wherein the chiral ruthenium catalyst comprises a chiral ligand of formula (IIIa), (IIIb), (IVa) or (IVb), where
Y is NR⁷ or O,
R⁴ is phenylsulphonyl, wherein the phenyl is unsubstituted or substituted with one or more substituents independently selected from C₁-C₄-alkyl and halogen, or
R⁴ is (2S)-2-pyrrolidinylcarbonyl,
R⁵ and R⁶ together form a —(CH₂)₄— group, or
R⁵ and R⁶ are unsubstituted phenyl,
R⁷ is hydrogen, phenyl-(CH₂)₃—, phenyl-(CH₂)₄—, benzyloxymethyl, benzyloxyethyl or phenyl-(CH₂)₂—O—CH₂— group, wherein the phenyl and benzyl groups are optionally substituted with one or more substituents independently selected from C₁-C₄-alkyl,
R⁸ and R⁹ are unsubstituted phenyl, or
R⁸ and R⁹ form together a group of formula 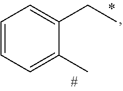
wherein the bond marked with “*” is connected to the carbon bearing the hydroxyl group and the bond marked with “#” is connected to the carbon bearing the amino group.

5. The process according to claim 2, wherein the chiral ruthenium catalyst comprises a chiral ligand of formula (IIIa) or (IIIb).

6. The process according to claim 1, wherein the compounds of formulae (Ia), (Ib) and (II) are compounds of formulae (Ia′), (Ib′) and (II′) 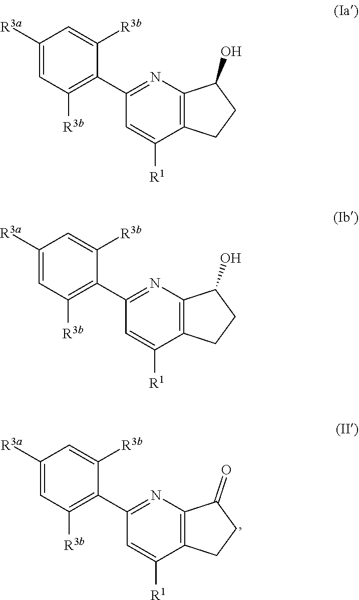
wherein R¹, R^{3a} and R^{3b} are independently of one another selected from C₁-C₄-alkyl.

7. The process according to claim 6, wherein
R¹ is methyl,
R^{3a} is methyl, and
R^{3b} is ethyl.

8. The process according to claim 1, wherein the hydrogen source is selected from the group consisting of sodium formate, potassium formate, lithium formate, calcium formate, magnesium formate, formic acid/triethylamine, potassium tert-butylate/isopropanol, sodium tert-butylate/isopropanol and lithium tert-butylate/isopropanol.

9. The process according to claim 1, wherein the hydrogen source is sodium formate or formic acid/triethylamine.

10. The process according to claim 1, wherein the amount of ruthenium catalyst used is within a range of from 0.1 mol % to 5 mol %, based on amount of the compound of formula (II).

11. The process according to claim 1, wherein transfer hydrogenation is conducted at a temperature within a range of from 20° C. to 80° C.

12. The process according to claim 1, wherein the polar solvent is selected from the group consisting of dichloromethane, methanol, ethanol, isopropanol, n-butanol, tetrahydrofuran, 2-methyl-tetrahydrofuran, dimethylformamide, acetonitrile, methanol/water, ethanol/water, isopropanol/water, n-butanol/water, tetrahydrofuran/water, 2-methyl-tetrahydrofuran/water, dimethylformamide/water, acetonitrile/water, and mixtures thereof.

13. The process according to claim 3, wherein R¹² is C₁-C₄-alkyl and Z is O or NH, and wherein the chiral ruthenium catalyst is formed in situ by mixing a dichloro (aromatic ligand)ruthenium (II) dimer precatalyst or a dibromo (aromatic ligand)ruthenium (II) dimer precatalyst with a chiral ligand of formula (IIIa′), (IIIb′), (IVa) or (IVb), 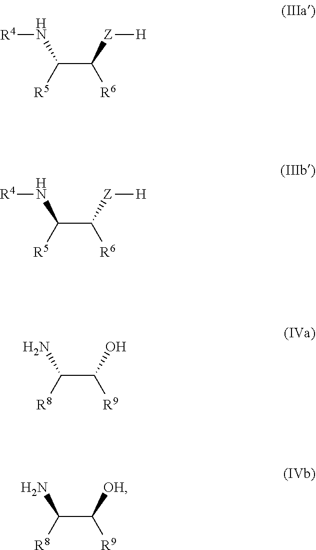
wherein
R⁴, R⁵ and R⁶ are each as defined for the complexes of formulae (Va) and (Vb),
Z is NH or O,
R⁸ and R⁹ are each as defined for the complexes of formulae (VIa) and (VIb),
and wherein the aromatic ligand of the precatalyst is selected from the group consisting of p-cymene and benzene, which is optionally substituted with one or more methyl groups,
in an organic solvent.

14. The process according to claim 1, wherein the product of formula (Ia) or (Ib) or a mixture thereof is purified by forming a crystalline addition salt with camphor sulfonic acid.

15. The process according to claim 1, wherein the ketone of formula (II) is obtained from a racemic mixture of the compounds of formulae (Ia) and (Ib) 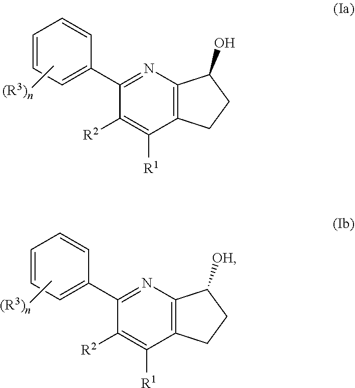
wherein the substituents R¹, R², R³ and the integer n are each as defined for the compound of formula (II),
by oxidation using TEMPO, a TEMPO derivative or a TEMPO analogue, a hypochlorite salt and optionally a bromide salt.
